SEQUENCE LISTING (7 sequences)

SEQ ID NO: 1 - DNA, 27 bp, Artificial Sequence
  Description of Artificial Sequence Synthetic oligonucleotide
rrbnnmnnnnnnnnnnnnnnnrrbnnm

SEQ ID NO: 2 - DNA, 2700 bp, Gallus gallus
  CDS (328)..(1482)
  modified_base (2237): unknown nucleotide
agcgcgccgccacccccgcaccctcccggggtcccgagcgcctcggctcgcggggagagctccttcttcggctcatttcgtttctttagtattatttttcgcctttcagccctcgcgtttcgtctcggttttctttttttttcttttatttttttcttttttttttcctcgcgtccccgggcctcggaagcacctcgcggcgaggatgtgtcggccccagcgggcgaagcagcgctgagaggccgaaggaggcacccgcccgcccgccgcccccggggggggggcacagcgctgcgccccgacggaccggctcccgcggctccgagcatgagcaagccggcgggatcaacaagtcgcattttggacatcccctgcaaagtgtgcggggaccgcagctccggcaagcactatggggtgtacgcctgcgacggctgctcgggtttcttcaagcggagcattaggaggaacaggacctacgtctgcaagtcgggaaatcaggggggctgtccggtggacaagacgcacaggaaccagtgccgggcctgccggctgaagaagtgcttggaagtcaacatgaacaaagacgctgtgcagcacgagcggggcccacgtacatcaacaatacggaagcaggtggccctctacttccgtggacacaaggaggagagcagcggtgccccgcactttcctgccactgccctgccagcacccgctttcttcactgctgtctcccagctggagccccatggcctggagctagctgcagttgccggcactcccgagaggcaggccctcgtgggcctggcgcagcccaccccaaagtacccgcatgaagtcaatggtacccccatgtatctctacgaggtggccaccgaatctgtctgtgagtcagcagccagacttctgttcatgagcatcaagtgggctaagagtgtaccggccttctctaccctgtccttacaagaccagctgatgcttttggaagatgcttggagagaactgtttgttctaggaatagcacaatgggccattccagttgatgctaacactctactggctgtatctggcatgaacggtgacaacacagattctcagaagctgaataaaatcatttcagaaatccaggctttacaggaggttgtggctagatttagacagctccggctagatgctactgaatttgcctgtctcaaatgcatcgtcactttcaaagctgtgccgacacacagcgggtccgagctgaggagcttccggaatgccgctgccatcgccgccctccaggacgaggcccagctcaccctgaacagctacatccataccaggtaccctacacaaccctgtcgtttcggaaaacttttattgcttctaccagctttacgttccattagtccatctacaatagaagaagtgtttttcaaaaagaccatcgggaatgtaccaatcacaagactgctttcagatatgtacaaatccagtgacatataaatttccttaaaacaaacgatcaggatggacagtttcagaagaacttttatctatggagaataagcctcaactaacaaaacctacaggaagcataagcctgggaatgtttagcctttaaacccattgacaaaaaataccccagtagatatgattctgctgttctgaacagagcgatttagatcatggagagaatgctttgttctacagaaaaagtgaaagtggttggaatttggctgcttttcctcttactacaggatggaggcaattcagatgccagtcatagttaacaaagactgcgctattctctcattgaactggcagatctgagacgaaatgtgaaagaaggtactttacttttgttcagtatttggaactgggtgaccaaacttggcttctgttgtaacgtgagatgtggtggccttcagaactgttttaaaagtagcctatgttggtaaaatgttttaatacgataggcaacatttaagaccgggttaccaaaacattgcttctgcagtaacacatcatgaagtggccttcagaactgattaaaaagtagcttatgacagcagttccactcttcctgcaaaataaactggtgatctgtgatgcagatgtcaccagaattaacatctcatgacagaagagctttatacaactccccgacccttctctcctccttacacacacgcacccacccacacagcccccgcaaagagtacacgtcaaggaaggaagacaaaaacagcaaaacnaaatccaatggagatgactgcttcagtgacctgctggctgtcccgttggcacactgctgaaaccaaaggcaacggcggccaaactctttgtcagtgagatgtgcagagaagtgaaatgactgtaaagacaccgacggcaacaattttatttcaaggaaataaagttggttgactgaaacgatgctaacaactttcaaaagtctccaatgcctttttagaaaactccaggttctaattttgaagccttgttcacctacaccctcttacacacaaaaatgttataagctgcttatttgatgtatgaaacacgtagaaaaaaaattaaagatagctgctgtacttttcaaaatttgatttgttattagaaactagcactgagaaaaatcagcacttggactatcatagaatgagtaaaacttttcctgtacaaagtggattaactgatttgtgaagttaaaaagtt

SEQ ID NO: 3 - protein, 385 aa, Gallus gallus
MSKPAGSTSRILDIPCKVCGDRSSGKHYGVYACDGCSGFFKRSIRRNRTYVCKSGNQGGCPVDKTHRNQCRACRLKKCLEVNMNKDAVQHERGPRTSTIRKQVALYFRGHKEESSGAPHFPATALPAPAFFTAVSQLEPHGLELAAVAGTPERQALVGLAQPTPKYPHEVNGTPMYLYEVATESVCESAARLLFMSIKWAKSVPAFSTLSLQDQLMLLEDAWRELFVLGIAQWAIPVDANTLLAVSGMNGDNTDSQKLNKIISEIQALQEVVARFRQLRLDATEFACLKCIVTFKAVPTHSGSELRSFRNAAAIAALQDEAQLTLNSYIHTRYPTQPCRFGKLLLLLPALRSISPSTIEEVFFKKTIGNVPITRLLSDMYKSSDI

SEQ ID NO: 4 - protein, 385 aa, Mus sp.
MSKPAGSTSRILDIPCKVCGDRSSGKHYGVYACDGCSGFFKRSIRRNRTYVCKSGNQGGCPVDKTHRNQCRACRLKKCLEVNMNKDAVQHERGPRTSTIRKQVALYFRGHKEDNGAAAHFPSTALPAPAFFTAVTQLEPHGLELAAVSATPERQTLVSLAQPTPKYPHEVNGTPMYLYEVATESVCESAARLLFMSIKWAKSVPAFSTLSLQDQLMLLEDAWRELFVLGIAQWAIPVDANTLLAVSGMNTDNTDSQKLNKIISEIQALQEVVARFRQLRLDATEFACLKCIVTFKAVPTHSGSELRSFRNAAAIAALQDEAQLTLNSYIHTRYPTQPCRFGKLLLLLPALRSISPSTIEEVFFKKTIGNVPITRLLSDMYKSSDI

SEQ ID NO: 5 - DNA, 26 bp, Mus sp.
gggcttgatgaagtcaagtccagtct

SEQ ID NO: 6 - DNA, 17 bp, Artificial Sequence
  Description of Artificial Sequence Synthetic GAL4 response element
cggaggactgtcctccg

SEQ ID NO: 7 - DNA, 26 bp, Homo sapiens
gggcttgatgaagtcaagtcgagtct

(19) United States
(12) Patent Application Publication
(10) Pub. No.: US 2006/0040321 A1
(43) Pub. Date: Feb. 23, 2006

(54) Orphan receptor TLX and uses therefor

(75) Inventors: Yanghong Shi, Arcadia, CA (US); Ruth T. Yu, La Jolla, CA (US); Fred H. Gage, La Jolla, CA (US); Ronald M. Evans, La Jolla, CA (US)

Correspondence Address: FOLEY & LARDNER LLP, P.O. BOX 80278, SAN DIEGO, CA, 92138-0278, US

(73) Assignee: The Salk Institute for Biological Studies

(21) Appl. No.: 11/096,072

(22) Filed: Mar. 31, 2005

Related U.S. Application Data
(60) Provisional application No. 60/558,593, filed on Mar. 31, 2004.

(51) Int. Cl. G01N 33/53; C07H 21/04; C07K 14/705; C12N 5/08

(52) U.S. Cl. 435/7.1; 435/320.1; 435/368; 530/350; 536/23.5

(57) ABSTRACT

In accordance with the present invention, there are provided methods for identifying compounds which modulate the activity of TLX. Such compounds will find use in a wide variety of applications, e.g., methods for relieving TLX-mediated transcription repression and/or inducing processes mediated by TLX, methods for inhibiting processes mediated by TLX, methods for promoting stem cell differentiation in a system in need thereof, and the like. In accordance with additional aspects of the present invention, there are provided methods for identifying compounds which modulate the expression of TLX. Such compounds will find use in a variety of applications, e.g., methods for treating neurodegenerative diseases in a subject in need thereof; methods for reversing the reduction of neurogenesis from neural stem cells in a subject in need thereof; methods for promoting generation of neural cell populations in a subject in need thereof; methods for maintaining adult neural stem cells in an undifferentiated, proliferative state; methods for rescuing neural stem cell activity in a system in need thereof; methods for promoting neural stem cell activity in a system in need thereof; and the like.

### RELATED APPLICATION

This application claims priority from U.S. Patent Application No. 60/558,593 filed Mar. 31, 2004, the entire contents which is hereby incorporated by reference herein.

### ACKNOWLEDGEMENT

This invention was made with Government support under Grant Number 5RO1-HD0 27183, awarded by the National Institutes of Health. The Government has certain rights in this invention.

### FIELD OF THE INVENTION

The present invention relates to members of the nuclear receptor superfamily and uses therefor. In a particular aspect, the present invention relates to the orphan receptor, TLX, and uses therefor.

### BACKGROUND OF THE INVENTION

The discovery of adult neural stem cells (NSCs) raises questions as to the nature and identification of the molecules that determine the self-renewal and multipotentiality of these cells. TLX was initially identified as an orphan nuclear receptor that is expressed in vertebrate forebrains (see Yu, R. T., McKeown, M., Evans, R. M. & Umesono, K. “Relationship between Drosophila gap gene tailless and a vertebrate nuclear receptor Tlx” in Nature 370, 375-9 (1994)). TLX expression in the mouse starts at embryonic day 8 (E8), peaks at E13.5, and decreases by E16, with barely detectable levels at birth. The expression of TLX increases after birth, with high levels in the adult brains (see Monaghan, A. P., Grau, E., Bock, D. & Schutz, G. “The mouse homolog of the orphan nuclear receptor tailless is expressed in the developing forebrain” in Development 121, 839-53 (1995)). While TLX null mice appear grossly normal at birth, the mature mice manifest a rapid retinopathy (see Yu, R. T. et al. “The orphan nuclear receptor Tlx regulates Pax2 and is essential for vision” in Proc Natl Acad Sci USA 97, 2621-5 (2000)) with reduced cerebral hemispheres (see Chiang, M. Y. & Evans, R. M. “Reverse Genetic Analysis of Nuclear Receptors, RXRg, RARb, and TLX in Mice” Dissertation (Univ. of California San Diego, La Jolla, Calif.) (1997), and Monaghan, A. P. et al. “Defective limbic system in mice lacking the tailless gene” in Nature 390, 515-7 (1997)).

Histologically, adult mutant brains have severely reduced hippocampal dentate gyri (DG), expanded lateral ventricles, and reduced olfactory bulb (OB), all of which are active adult neurogenic areas (see Gage, F. H. “Neurogenesis in the adult brain” in J Neurosci 22, 612-3 (2002)).

Behaviorally, TLX mutants exhibit increased aggressiveness, decreased copulation, and progressively violent behavior (see Chiang & Evans, supra and Monaghan, supra.). The hypomorphic defects and neurological disorders of the mutant mice suggest a role for TLX in normal CNS function.

Accordingly, identification of the role of TLX in normal CNS function would be of great interest, and would make available new therapeutic approaches to the treatment of CNS disorders.

### SUMMARY OF THE INVENTION

The recent finding of neurogenesis in the adult brain has led to the discovery of adult neural stem cells (NSCs) (see Taupin, P. and Gage, F. H., “Adult neurogenesis and neural stem cells of the central nervous system in mammals” in J. Neurosci. Res. 69, 745-749 (2002) and Gage, F. H. “Mammalian neural stem cells” in Science 287, 1433-8 (2000)). Neural stem cells are the self-renewing, multipotent cells that generate neurons, astrocytes, and oligodendrocytes in the nervous system. Over the past decades, the confirmation that neurogenesis occurs in discrete areas of the adult brain and that NSCs reside in the adult brain has overturned the long-held dogma that we are born with a certain number of nerve cells and that the brain cannot generate new neurons and renew itself. Neurogenesis has been shown to occur throughout adulthood in two neurogenic areas of the adult mammalian CNS: the olfactory bulb (OB) and the dentate gyrus (DG) of the hippocampus. Low levels of neurogenesis have also been reported in the Ammon's horn of the adult mouse. Neurogenesis has been shown to occur in the OB of adult rodents and non-human primates.

NSC research has focused on identifying the NSCs of the adult CNS. The first cells from the adult CNS characterized as capable of generating the three main phenotypes of the CNS in vitro were isolated from mouse striatal tissue. These putative NSCs were called neural progenitor cells (NPCs) because their stem cell properties had yet to be demonstrated. The NPCs were found to be immunoreactive for the intermediate filament protein nestin and to give rise to neuronal and glial cells, astrocytes and oligodendrocytes in vitro. Nestin has been characterized as a marker for neuroepithelial and CNS stem cells in vitro and in vivo. NPCs have since been isolated from diverse areas of the adult CNS: in mouse brain, in the subventricular zone (SVZ) of mouse, rat and human; in rat and human hippocampus; in rat septum and striatum; in human cortex; in human and mouse OB; in the rostral extension of the mouse SVZ; and in different levels of the spinal cord (cervical, thoracic, lumbar, and sacral) of the mouse and rat. In the spinal cord, NPCs can be isolated from the periventricular area and the parenchyma. NPCs have also been isolated and cultured from adult postmortem brain tissues. NPCs have been isolated and cultured after postmortem intervals of up to 140 hr from adult mouse SVZ and spinal cord, adult human OB, and human hippocampus and SVZ.

The demonstration that multipotent, self-renewing progenitor cells of neurons and glial cells can be cultured from NPCs from these adult brain regions shows that NPC cultures contain some NSCs, and demonstrating that NPCs are multipotent relies on evidence that neurons, astrocytes and oligodendrocytes, the three main phenotypes of the CNS, can be generated from single cells. The demonstration that NPCs can self-renew relies on showing that NPCs maintain their multipotentiality over time. Adult-derived NSCs have now been characterized from other brain regions, such as the hippocampus, the spinal cord and the SVZ, and from different species, including rodents and humans. These studies confirm the existence of NSCs in the adult CNS and show that NSCs, like NPCs, can be isolated from neurogenic and non-neurogenic areas.

Neurogenesis occurs constitutively throughout adulthood in the SVZ and the DG, but it has been reported that the rate of neurogenesis decreases with age in rodents. The decrease in neurogenesis maybe due to a decrease in the number of NSCs and/or NPCs. Studies in the rodent SVZ have demonstrated that NPCs can be isolated and cultured from aged SVZ with the same efficiency as younger SV. In addition or alternatively, the decrease in neurogenesis observed with aging may be to a progressive lengthening of the cell cycle time of the NPCs in vivo.

In accordance with the present invention, it is shown that the orphan nuclear receptor TLX (see Yu, McKeown, Evans & Umesono, supra) maintains adult NSCs in an undifferentiated, proliferative state. It is also demonstrated herein that the TLX-expressing cells isolated by FACS from adult brains are able to proliferate, self-renew, and differentiate into all neural cell types in vitro. In contrast, the TLX^{-/-} cells isolated from adult mutant brains fail to proliferate. Reintroduction of TLX into the FACS-sorted TLX^{-/-} cells rescues the ability to proliferate and self-renew. In vivo, the TLX mutant mice display a loss of cell proliferation and a significant reduction in nestin labeling in the adult neurogenic areas. Finally, TLX has been found to be capable of silencing glia-specific GFAP expression in NSCs, suggesting that transcriptional repression may be crucial in maintaining their undifferentiated state.

In accordance with another aspect of the invention, there are provided methods for identifying compounds which modulate the activity of TLX. Such compounds will find use in a wide variety of applications, e.g., methods for relieving TLX-mediated transcription repression and/or inducing processes mediated by TLX, methods for inhibiting processes mediated by TLX, methods for promoting stem cell differentiation in a system in need thereof, and the like.

In accordance with yet another aspect of the present invention, there are provided methods for identifying compounds which modulate the expression of TLX. Such compounds will find use in a variety of applications, e.g., methods for treating neurodegenerative diseases in a subject in need thereof; methods for reversing age-related depopulation of neural stem cells in a subject in need thereof; methods for rescuing degenerated neural cell populations in a subject in need thereof; methods for promoting generation of neural cell populations in a subject in need thereof; methods for maintaining adult neural stem cells in an undifferentiated, proliferative state; methods for rescuing neural stem cell activity in a system in need thereof; methods for promoting neural stem cell activity in a system in need thereof; and the like.

### BRIEF DESCRIPTION OF THE FIGURES

FIG. 1 presents the complete nucleotide and predicted amino acid sequences for TLX. The predicted amino acid sequence of chick TLX is aligned with that of mouse TLX. Identical amino acids are indicated by dashed lines and substitutions are boxed. Solid arrows demarcate the DNA binding domain (DBD) and open arrows demarcate the ligand binding domain (LBD).

FIG. 2 illustrates the effect of TLX alone, TLX-EnR (i.e., a fusion of TLX and the engrailed repressor domain; see Yu, R. T. et al. “The orphan nuclear receptor Tlx regulates Pax2 and is essential for vision” in Proc Natl Acad Sci USA 97, 2621-5 (2000)) and TLX-VP (i.e., a fusion of TLX and the VP16 activation domain; see Yu et al., supra) on the Pax2 promoter. While TLX alone and TLX-EnR repress the Pax2 promoter, TLX-VP acts as a dominant negative factor, thereby de-repressing Pax2 transcription.

### DETAILED DESCRIPTION OF THE INVENTION

In accordance with the present invention, there are provided methods for screening compounds to determine those which modulate the activity of TLX, said methods comprising contacting a test cell with one or more test compounds, and assaying for evidence of transcription of reporter by said test cells,

wherein said test cell expresses TLX and contains a reporter construct comprising a TLX-response element operatively linked to a reporter gene.

As used herein, the phrase “assaying for evidence of transcription” refers to well-known methods for detecting the various products of transcription, such as mRNA or the corresponding amino acid sequence. Exemplary methods for detecting evidence of transcription include, for example, the cis/trans assay described in U.S. Pat. Nos. 5,171,671 and 4,981,784, (each of which are incorporated herein by reference), and the like.

As employed herein, the term “modulate” refers to the ability of a modulator for TLX to directly (by binding to the receptor as a ligand) or indirectly (as a precursor for a ligand or an inducer which promotes production of ligand from a precursor) induce expression of gene(s) maintained under hormone expression control, or to directly or indirectly repress expression of gene(s) maintained under such control.

Any cell line can be used as a suitable “test cell” for the functional bioassay contemplated for use in the practice of the present invention. Thus, cells contemplated for use in the practice of the present invention include transformed cells, non-transformed cells, neoplastic cells, primary cultures of different cell types, and the like. Exemplary cells which can be employed in the practice of the present invention include Schneider cells, CV-1 cells, HuTu80 cells, F9 cells, NTERA2 cells, NB4 cells, HL-60 cells, 293 cells, Hela cells, yeast cells, and the like. Preferred host cells for use in the functional bioassay system are COS cells and CV-1 cells. COS-1 (referred to as COS) cells are monkey kidney cells that express SV40 T antigen (Tag); while CV-1 cells do not express SV40 Tag. The presence of Tag in the COS-1 derivative lines allows the introduced expression plasmid to replicate and provides a relative increase in the amount of receptor produced during the assay period. CV-1 cells are presently preferred because they are particularly convenient for gene transfer studies and provide a sensitive and well-described host cell system.

In accordance with a still further embodiment of the present invention, there are provided expression vectors encoding TLX, operatively associated with a suitable promoter. As employed herein, “expression vector” means a vector which is capable of effecting expression of a DNA sequence contained in the vector once the vector has been transfected, transformed, microinjected or otherwise introduced into a suitable cell. In an expression vector, the DNA sequence to be expressed is operatively linked to other sequences capable of effecting the transcription of the DNA sequence along with other sequences, such that the transcript of the DNA sequence can be productively translated. A “suitable cell” for the vector is one in which these other, transcription-effecting sequences and the resulting translation-effecting sequences are recognized for transcription and translation. Construction of an expression vector of the invention and for use in accordance with the present invention is well within the skill of the person of ordinary skill in molecular biology, as are methods of introducing such a vector into cells suitable for expression of the DNA sequence intended to be expressed with the vector. An expression vector, in a suitable cell in which the vector is operative, can function as an episome or can be integrated into genomic DNA of the cell. An expression vector can be a circularized plasmid, a linearized plasmid or a part thereof, or all or part of a viral genome. Preferred for the present invention are expression vectors that are operative to effect expression of a DNA sequence in mammalian cells.

An expression vector includes elements capable of expressing DNAs that are operatively linked with regulatory sequences (such as promoter regions) that are capable of regulating expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are those that are replicable in eukaryotic cells and/or prokaryotic cells, including those that remain episomal or those which integrate into the host cell genome.

Exemplary eukaryotic plasmid expression vectors include eukaryotic cassettes, such as the pSV-2 gpt system (Mulligan et al., 1979, Nature 277:108-114) and the expression cloning vector described by Genetics Institute (1985, Science 228:810-815). These plasmid vectors, when modified to contain an invention DNA construct, are able to provide at least some expression of the protein of interest in response to a retinoid, or the like.

Other plasmid base vectors which contain regulatory elements that can be operatively linked to the invention response elements are cytomegalovirus (CMV) promoter-based vectors such as pcDNA1 (Invitrogen, San Diego, Calif.), MMTV promoter-based vectors such as pMAMNeo (Clontech, Palo Alto, Calif.) and pMSG (Pharnacia, Piscataway, N.J.), and SV40 promoter-based vectors such as pSVP (Clontech, Palo Alto, Calif.).

The above-described cells (or fractions thereof) are maintained under physiological conditions. “Physiological conditions” are readily understood by those of skill in the art to comprise an isotonic, aqueous nutrient medium at a temperature of about 37° C.

As readily recognized by those of skill in the art, a wide variety of test compounds can be employed in the practice of the present invention. Compounds contemplated for screening in accordance with the present invention may be obtained from well-known sources, e.g., from combinatorial chemical libraries, peptide libraries, chemical libraries, bacterial and yeast broths, plants, and the like.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250). Preparation and screening of combinatorial chemical libraries are well known to those of skill in the art, see, e.g., U.S. Pat. Nos. 6,004,617; 5,985,356. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), non-peptidal peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. No. 6,045,755; 5,792,431; 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Once suitable lead compounds have been identified, rational drug design methods can be used to optimize the utility of the compound as a biopharmaceutical agent. As used herein, “rational drug design” refers to any of a variety of methods which can be used to design candidate compounds based on structural and/or functional information derived from lead compounds. In such methods, for example, 3-dimensional structure information obtained from x-ray crystallographic or NMR studies of a target polypeptide can be used to specifically produce or modify a therapeutic agent to interact more specifically and/or effectively with the wildtype protein target, thus increasing the therapeutic efficacy of the parental drug and/or decreasing non-specific, potentially deleterious interactions. See, e.g., Hicks, Curr. Med. Chem. 8: 627-50 (2001); Gane and Dean, Curr. Opin. Struct. Biol. 10: 401-4 (2000).

Examples of compounds contemplated for screening in accordance with the present invention, include, for example, small molecules, alkaloids and other heterocyclic organic compounds, and the like.

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, iron, potassium, nitrogen, oxygen, sulfur, and other elements. An organic compound may be aromatic or aliphatic. Non-limiting examples of organic compounds include alcohols, aldehydes, ketones, carbohydrates, monosaccharides, oligosaccharides, polysaccharides, amino acids, nucleosides, nucleotides, lipids, retinoids, steroids, proteoglycans, saturated, unsaturated and polyunsaturated fats, fatty acids, oils and waxes; alkenes, esters, ethers, thiols, sulfides, cyclic compounds (e.g., phenols, anilines, and the like), heterocylcic compounds (e.g., imidazoles, purines, pyrimidines, and the like), and the like. An organic compound as used herein also includes nitrated organic compounds and halogenated (e.g., chlorinated) organic compounds. Collections of small molecules, and small molecules identified according to the invention can be characterized by a variety of techniques such as accelerator mass spectrometry (AMS; see Turteltaub et al., Curr Pharm Des 2000 6(10):991-1007, Bioanalytical applications of accelerator mass spectrometry for pharmaceutical research; and Enjalbal et al., Mass Spectrom Rev 2000 19(3):139-61, Mass spectrometry in combinatorial chemistry).

Preferred small molecules contemplated for use in the practice of the present invention are relatively easily and inexpensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

Reporter constructs contemplated for use herein can be any plasmid which contains an operative TLX-response element functionally linked to an operative reporter gene. Exemplary reporter genes include β-galactosidase, luciferase, Green fluorescent protein (GFP), chloramphenicol acetyltransferase (CAT), and the like.

TLX response elements contemplated for use in the practice of the present invention are based on the consensus TLX binding site: AAGTCA. Such response elements can be composed of two or more “half sites”, wherein each half site comprises the sequence -RRBNNM-,

wherein:

each R is independently selected from A or G;

each B is independently selected from G, C, or T;

each N is independently selected from A, T, C, or G; and

each M is independently selected from A or C; with the proviso that at least 4 nucleotides of each -RRBNNM- group of nucleotides are identical with the nucleotides at comparable positions of the sequence -AAGTCA-.

Thus, exemplary response elements can be represented as follows:
5′-RRBNNM-[(N)ₓ-RRBNNM]_{y}-3′ (SEQ ID NO:1),
   wherein:

x is zero or a whole number up to 15, and

y is at least 1,

with the proviso that at least 4 of the nucleotides in the half-site sequence are identical with the nucleotides at comparable positions of the sequence -AAGTCA-. Where one of the half sites varies by 2 nucleotides from the preferred sequence of -AAGTCA-, it is preferred that the other half site of the response element be the same as, or vary from the preferred sequence by no more than 1 nucleotide. It is presently preferred that the 3′-half site (or downstream half site) of a pair of half sites vary from the preferred sequence by at most 1 nucleotide.

Exemplary response elements contemplated by the present invention are derived from various combinations of half sites having sequences selected from, for example, -AAGTCA-, -AGGTCA-, -GGGTCA-, -AAGTGA-, -AGGTGA-, -GGGTCA-, and the like.

As used herein, the phrase “operatively associated with” means that the respective DNA sequences (represented, for example, by the terms “TLX response element” and “reporter gene”) are operational, i.e., work for their intended purposes; the word “functionally” means that after the two segments are linked, upon appropriate activation by a ligand-receptor complex, the reporter gene will be expressed as the result of the fact that the corresponding “response element” was “turned on” or otherwise activated.

Screening assays may be low throughput screening assays, wherein no more than a limited number of compounds is run through a selective assay at one time in one or more iterations of the assay. In high throughput screening (HTS) assays, compounds are screened en masse; i.e., a large collection (at least a pool, preferably a library) of compounds are rapidly run through one or more selective assays, typically with the assistance of automated and semi-automated devices, especially library preparation devices and compound detection devices. Various methods for analyzing the interaction of a known or suspected ligand molecule with its preselected target are known. Such methods include without limitation assays that detect bound or unbound ligands, and/or unbound or bound target molecules. Such assays may utilize mass spectrometry, phosphorescence, chemiluminescence, luminescence, fluorescence polarization, resonance energy transfer, liquid chromatography; assays based on affinity capture and/or competitive inhibition of known or suspected ligands directed to a target molecule; assays that determine binding to the site (e.g., epitope) or measure the degree (e.g., Kd, Ki, etc.) of binding of known or suspected ligands for target molecules; and scintillation proximity assays. In most assays, at least one detectably labeled molecular reagent is used.

HTS techniques, devices and software are described in the following publications, which are incorporated herein by reference: Strege Mass., High-performance liquid chromatographic-electrospray ionization mass spectrometric analyses for the integration of natural products with modern high-throughput screening, Journal of Chromatography. B, Biomedical Sciences & Applications. 725:67-78, 1999; Grabley S. Thiericke R., Bioactive agents from natural sources: trends in discovery and application, Advances in Biochemical Engineering-Biotechnology. 64:101-54, 1999; Kenny B A. Bushfield M. Parry-Smith D J. Fogarty S. Treherne J M., The application of high-throughput screening to novel lead discovery, Progress in Drug Research. 51:245-69, 1998; Rodrigues A D., Preclinical drug metabolism in the age of high-throughput screening: an industrial perspective, Pharmaceutical Research. 14:1504-10, 1997; Humphery-Smith I. Cordwell S J. Blackstock W P., Proteome research: complementarity and limitations with respect to the RNA and DNA worlds, Electrophoresis 18:1217-42, 1997.

Kd may be measured in solution using techniques and compositions described in the following publications. Blake, D. A.; Blake, R. C.; Khosraviani, M.; Pavlov, A. R. “Immunoassays for Metal Ions.” Analytica Chimica Acta 1998, 376, 13-19. Blake, D. A.; Chakrabarti, P.; Khosraviani, M.; Hatcher, F. M.; Westhoff, C. M.; Goebel, P.; Wylie, D. E.; Blake, R. C. “Metal Binding Properties of a Monoclonal Antibody Directed toward Metal-Chelate Complexes.” Journal of Biological Chemistry 1996, 271(44), 27677-27685. Blake, D. A.; Khosraviani, M.; Pavlov, A. R.; Blake, R.C. “Characterization of a Metal-Specific Monoclonal Antibody.” Aga, D. S.; Thurman, E. M., Eds.; ACS Symposium Series 657; American Chemical Society: Washington, D.C., 1997; pp 49-60.

Kd is measured using immobilized binding components on a chip, for example, on a BIAcore chip using surface plasmon resonance. Surface plasmon resonance is used to characterize the microscopic association and dissociation constants of reaction between sFv directed against pIgG associated molecules and pIgR and pIgR fragments. Such general methods are described in the following references and are incorporated herein by reference (Vely F. Trautmann A. Vivier E., BIAcore analysis to test phosphopeptide-SH2 domain interactions, Methods in Molecular Biology. 121:313-21, 2000; Liparoto S F. Ciardelli T L., Biosensor analysis of the interleukin-2 receptor complex, Journal of Molecular Recognition. 12:316-21, 1999; Lipschultz C A. Li Y. Smith-Gill S., Experimental design for analysis of complex kinetics using surface plasmon resonance, Methods. 20):310-8, 2000; Malmqvist M., BIACORE: an affinity biosensor system for characterization of biomolecular interactions, Biochemical Society Transactions. 27:335-40, 1999; Alfthan K., Surface plasmon resonance biosensors as a tool in antibody engineering, Biosensors & Bioelectronics. 13:653-63, 1998; Fivash M. Towler E M. Fisher R J., BIAcore for macromolecular interaction, Current Opinion in Biotechnology. 9:97-101, 1998; Price M R. Rye P D. Petrakou E. Murray A. Brady K. Imai S. Haga S. Kiyozuka Y. Schol D. Meulenbroek M F. Snijdewint F G. Von Mensdorff-Pouilly S. Verstraeten R A. Kenemans P. Blockzjil A. Nilsson K. Nilsson O. Reddish M. Suresh M R. Koganty R R. Fortier S. Baronic L. Berg A. Longenecker M B. Hilgers J. et al.; Summary report on the ISOBM TD-4 Workshop: analysis of 56 monoclonal antibodies against the MUCI mucin. San Diego, Calif., Nov. 17-23, 1996, Tumour Biology. 19 Suppl 1: 1-20, 1998; Malmqvist M. Karlsson R, Biomolecular interaction analysis: affinity biosensor technologies for functional analysis of proteins, Current Opinion in Chemical Biology. 1:378-83, 1997; O'Shannessy D J. Winzor D J., Interpretation of deviations from pseudo-first-order kinetic behavior in the characterization of ligand binding by biosensor technology, Analytical Biochemistry. 236:275-83, 1996; Malmborg A C. Borrebaeck C A, BIAcore as a tool in antibody engineering, Journal of Immunological Methods. 183:7-13, 1995; Van Regenmortel M H., Use of biosensors to characterize recombinant proteins, Developments in Biological Standardization. 83:143-51, 1994; O'Shannessy D J., Determination of kinetic rate and equilibrium binding constants for macromolecular interactions: a critique of the surface plasmon resonance literature, Current Opinion in Biotechnology. 5:65-71, 1994).

In accordance with another aspect of the present invention, there are provided compounds identified by the above-described methods. Such compounds can be either TLX agonists or TLX antagonists. As used herein, the term “agonist” refers to an agent or compound that enhances or increases the activity of a TLX polypeptide or polynucleotide. An agonist may be directly active on a TLX polypeptide or polynucleotide, or it may be active on one or more constituents in a pathway that leads to enhanced or increased activity of a TLX polypeptide or polynucleotide.

As used herein, the term “antagonist” refers to an agent or compound that reduces or decreases the activity of a TLX polypeptide or polynucleotide. An antagonist may be directly active on a TLX polypeptide or polynucleotide, or it may be active on one or more constituents in a pathway that leads to reduced or decreased activity of a TLX polypeptide or polynucleotide. Antagonists can act mechanistically by either inhibiting ligand-binding to a respective receptor, or by inhibiting an activated ligand-receptor complex from binding to its respective DNA response element, or by inhibiting an activated ligand-receptor complex from binding to a cofactor required for the activation of transcription.

Compounds identified by the above-described methodology can be used in a variety of applications, e.g., methods for relieving TLX-mediated transcription repression and/or inducing processes mediated by TLX. Such methods comprise conducting processes mediated by TLX in the presence of a compound identified by the above-described method. Optionally, such processes can be conducted in the further presence of at least one inhibitor of a co-repressor.

Inhibitors of co-repressors contemplated for use herein include histone deacetylase inhibitors (e.g., Trichostatin A (TSA), Trapoxin, and the like), chromatin remodeling machinery inhibitors, and the like.

As used herein, the phrase “inhibit activation of transcription” refers to blocking the well known process whereby mRNA is transcribed from a respective cDNA coding sequence. The amount of mRNA transcription can be detected by a variety of methods well-known in the art, such as detecting levels of reporter protein expression, detecting directly the level of mRNA transcribed, and the like.

Additional uses for compounds identified by the above-described methodology include methods for inhibiting processes mediated by TLX. Such methods comprise conducting processes mediated by TLX in the presence of a compound identified by the above-described method.

Still another use for compounds identified by the above-identified methodology include methods for promoting stem cell differentiation in a system in need thereof. Such methods comprise contacting such a system with a compound identified by the above-described method.

An alternate method for promoting stem cell differentiation in a system in need thereof comprises blocking expression of TLX in said system. As readily recognized by those of skill in the art, expression of TLX can be blocked in a variety of ways, e.g., by use of antisense, ribozyme, and/or RNAi molecules, gene or regulatory sequence replacement constructs, decoy oligonucleotides, and the like.

Antisense oligonucleotides capable of binding polypeptide message can inhibit polypeptide activity by targeting mRNA. Strategies for designing antisense oligonucleotides are well disclosed in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Antisense oligonucleotides can be of any length; for example, in alternative aspects, the antisense oligonucleotides are between about 5 to 100 residues, about 10 to 80 residues, about 15 to 60 residues, or about 18 to 40 residues. The optimal length can be determined by routine screening. The antisense oligonucleotides can be present at any concentration. A wide variety of synthetic, non-naturally occurring nucleotide and nucleic acid analogues are known which can be employed as contemplated herein. For example, peptide nucleic acids (PNAs) containing non-ionic backbones, such as N-(2-aminoethyl)glycine units can be used. Antisense oligonucleotides having phosphorothioate linkages can also be used, as disclosed in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold (1995) J. of Biol. Chem. 270:13581-13584).

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RNaseP-like RNA (in association with an RNA guide sequence). Examples of such hammerhead motifs are disclosed by Rossi (1992) Aids Research and Human Retroviruses 8:183; hairpin motifs by Hampel (1989) Biochemistry 28:4929, and Hampel (1990) Nucl. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNA interference (“RNAi”) refers to methods by which double-stranded RNA molecules trigger a gene silencing response in various cells. In these methods, soluble-stranded RNA molecules are reduced to small interfering RNAs (“siRNAs”), preferably about 21-23 nucleotides in length, by endogenous nucleases. Methods have been disclosed for the design of RNAi oligonucleotides to provide sequence-specific gene silencing. See, e.g., Elbashir (2001) Nature 411:494. The RNAi phenomenon differs from antisense methods, in that it is mediated by double-stranded RNA rather than by single-stranded antisense RNA. Its use has been demonstrated in cells as diverse as those from the nematode C. elegans to numerous mammalian cell types.

RNAi oligonucleotides may be provided to cells either as presynthesized (by either in vitro or in vivo methods) double-stranded RNA molecules, and/or by expressing the RNAi oligonucleotide directly in target cells. For expression of siRNAs within cells, some researchers engineered plasmid vectors that contained either the polymerase III H1-RNA, or U6 promoter, a cloning site for the stem-looped RNA insert, and a 4-5-thymidine transcription termination signal. The inserts were ˜50 nucleotides (nt), with ˜20 nt inverted repeats (coding for the dsRNA stem complementary to a target gene) and ˜10 nt spacers (coding for the loop). Polymerase III promoters were chosen because these promoters generally have well-defined initiation and stop sites and their transcripts lack poly(A) tails. The termination signal for these promoters is defined by 5 thymidines, and the transcript is typically cleaved after the second uridine. Cleavage at this position generates a 3′ UU overhang in the expressed siRNA, which is similar to the 3′ overhangs of synthetic siRNAs. In another approach, U6 promoter-driven expression vectors were made that expressed the sense and antisense strands of siRNAs. Upon expression, these strands presumably anneal in vivo to produce the functional siRNAs. See, e.g., Brummelkamp (2002), Science 296:550; Paddison (2002), Genes and Dev. 16:948; Paul (2002), Nature Biotechnol. 20:505; Sui (2002), Proc. Natl. Acad. Sci. USA 99:5515. Yu (2002), Proc. Natl. Acad. Sci. USA 99:6047; Miyagishi and Taira (2002), Nature Biotechnol. 20:497; and Lee, (2002), Nature Biotechnol. 20:500.

“Decoy oligonucleotides” refer to double stranded nucleic acids that bind to a DNA binding protein, thereby preventing binding of the DNA binding protein to its natural target in the cell. Transfection of cis-element double stranded (ds) decoy oligonucleotides has been reported as a powerful tool for gene therapy. See, e.g., Tomita (1997), Exp. Nephrol. 5(5):429. The decoy approach may also enable us to treat diseases by modulation of endogenous transcriptional regulation as a “loss of function” approach at the pre-transcriptional and transcriptional levels in a similar fashion to employing antisense technology as a “loss of function” approach at the transcriptional and translational levels.

As employed herein, the phrase “biological system” refers to an intact organism or a cell-based system containing the various components required for response to the compounds described herein, e.g., an isoform TLX, a silent partner for TLX (e.g., RXR), and a TLX-responsive reporter (which typically comprises a TLX response element (TLX-RE) in operative communication with a reporter gene; suitable reporters include luciferase, chloramphenicol transferase, β-galactosidase, and the like.

Contacting in a biological system contemplated by the present invention can be accomplished in a variety of ways, and the treating agents contemplated for use herein can be administered in a variety of forms (e.g., in combination with a pharmaceutically acceptable carrier therefor) and by a variety of modes of delivery. Exemplary pharmaceutically acceptable carriers include carriers suitable for oral, sublingual, intravenous, subcutaneous, transcutaneous, intramuscular, intracutaneous, intrathecal, epidural, intraoccular, intracranial, inhalation, rectal, vaginal, and the like administration. Administration in the form of creams, lotions, tablets, dispersible powders, granules, syrups, elixirs, sterile aqueous or non-aqueous solutions, suspensions or emulsions, and the like, is contemplated.

The preferred route of administration will vary with the clinical indication. Some variation in dosage will necessarily occur depending upon the condition of the patient being treated, and the physician will, in any event, determine the appropriate dose for the individual patient. The effective amount of compound per unit dose depends, among other things, on the body weight, physiology, and chosen inoculation regimen. A unit dose of compound refers to the weight of compound employed per administration event without the weight of carrier (when carrier is used).

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use.

Targeted-delivery systems, such as polymer matrices, liposomes, and microspheres can increase the effective concentration of a therapeutic agent at the site where the therapeutic agent is needed and decrease undesired effects of the therapeutic agent. With more efficient delivery of a therapeutic agent, systemic concentrations of the agent are reduced because lesser amounts of the therapeutic agent can be administered while accruing the same or better therapeutic results. Methodologies applicable to increased delivery efficiency of therapeutic agents typically focus on attaching a targeting moiety to the therapeutic agent or to a carrier which is subsequently loaded with a therapeutic agent.

Various drug delivery systems have been designed by using carriers such as proteins, peptides, polysaccharides, synthetic polymers, colloidal particles (i.e., liposomes, vesicles or micelles), microemulsions, microspheres and nanoparticles. These carriers, which contain entrapped pharmaceutically useful agents, are intended to achieve controlled cell-specific or tissue-specific drug release.

The compounds described herein can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The compounds described herein, when in liposome form can contain, in addition to the compounds described herein, stabilizers, preservatives, excipients, and the like. The preferred lipids are the phospholipids and the phosphatidyl cholines (lecithins), both natural and synthetic. Methods to form liposomes are known in the art. (See, e.g., Prescott, Ed., Methods in Cell Biology, Volume XIV, Academic Press, New York, N.Y., (1976), p 33 et seq.)

Several delivery approaches can be used to deliver therapeutic agents to the brain by circumventing the blood-brain barrier. Such approaches utilize intrathecal injections, surgical implants (Ommaya, Cancer Drug Delivery, 1: 169-178 (1984) and U.S. Pat. No. 5,222,982), interstitial infusion (Bobo et al., Proc. Natl. Acad. Sci. U.S.A., 91: 2076-2080 (1994)), and the like. These strategies deliver an agent to the CNS by direct administration into the cerebrospinal fluid (CSF) or into the brain parenchyma (ECF).

Drug delivery to the central nervous system through the cerebrospinal fluid is achieved, for example, by means of a subdurally implantable device named after its inventor the “Ommaya reservoir”. The drug is injected into the device and subsequently released into the cerebrospinal fluid surrounding the brain. It can be directed toward specific areas of exposed brain tissue which then adsorb the drug. This adsorption is limited since the drug does not travel freely. A modified device, whereby the reservoir is implanted in the abdominal cavity and the injected drug is transported by cerebrospinal fluid (taken from and returned to the spine) to the ventricular space of the brain, is used for agent administration. Through omega-3 derivatization, site-specific biomolecular complexes can overcome the limited adsorption and movement of therapeutic agents through brain tissue.

Another strategy to improve agent delivery to the CNS is by increasing the agent absorption (adsorption and transport) through the blood-brain barrier and the uptake of therapeutic agent by the cells (Broadwell, Acta Neuropathol., 79: 117-128 (1989); Pardridge et al., J. Pharmacol. Experim. Therapeutics, 255: 893-899 (1990); Banks et al., Progress in Brain Research, 91:139-148 (1992); Pardridge, Fuel Homeostasis and the Nervous System, ed.: Vranic et al., Plenum Press, New York, 43-53 (1991)). The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier, or by its covalent coupling to a peptide vector capable of transporting the agent through the blood-brain barrier. Peptide transport vectors are also known as blood-brain barrier permeabilizer compounds (U.S. Pat. No. 5,268,164). Site specific macromolecules with lipophilic characteristics useful for delivery to the brain are described in U.S. Pat. No. 6,005,004.

Other examples (U.S. Pat. No.4,701,521, and U.S. Pat. No.4,847,240) describe a method of covalently bonding an agent to a cationic macromolecular carrier which enters into the cells at relatively higher rates. These patents teach enhancement in cellular uptake of bio-molecules into the cells when covalently bonded to cationic resins.

U.S. Pat. No. 4,046,722 discloses anti-cancer drugs covalently bonded to cationic polymers for the purpose of directing them to cells bearing specific antigens. The polymeric carriers have molecular weights of about 5,000 to 500,000. Such polymeric carriers can be employed to deliver compounds described herein in a targeted manner.

Further work involving covalent bonding of an agent to a cationic polymer through an acid-sensitive intermediate (also known as a spacer) molecule, is described in U.S. Pat. No. 4,631,190 and U.S. Pat. No. 5,144,011. Various spacer molecules, such as cis-aconitic acid, are covalently linked to the agent and to the polymeric carrier. They control the release of the agent from the macromolecular carrier when subjected to a mild increase in acidity, such as probably occurs within a lysosome of the cell. The drug can be selectively hydrolyzed from the molecular conjugate and released in the cell in its unmodified and active form. Molecular conjugates are transported to lysosomes, where they are metabolized under the action of lysosomal enzymes at a substantially more acidic pH than other compartments or fluids within a cell or body. The pH of a lysosome is shown to be about 4.8, while during the initial stage of the conjugate digestion, the pH is possibly as low as 3.8.

As employed herein, the phrase “effective amount” refers to levels of compound sufficient to provide circulating concentrations high enough to modulate the expression of gene(s) mediated by TLX. Such a concentration typically falls in the range of about 10 nM up to 2 μM; with concentrations in the range of about 100 nM up to 500 nM being preferred. Since the activity of different compounds described herein may vary considerably, and since individual subjects may present a wide variation in severity of symptoms, it is up to the practitioner to determine a subject's response to treatment and vary the dosages accordingly.

In accordance with another embodiment of the present invention, there are provided methods for screening compounds to determine those which modulate the expression of TLX, said method comprising contacting a test cell with one or more test compounds, and assaying for evidence of transcription of reporter by said test cells, wherein said test cell contains a reporter construct comprising a TLX promoter operatively linked to a reporter gene.

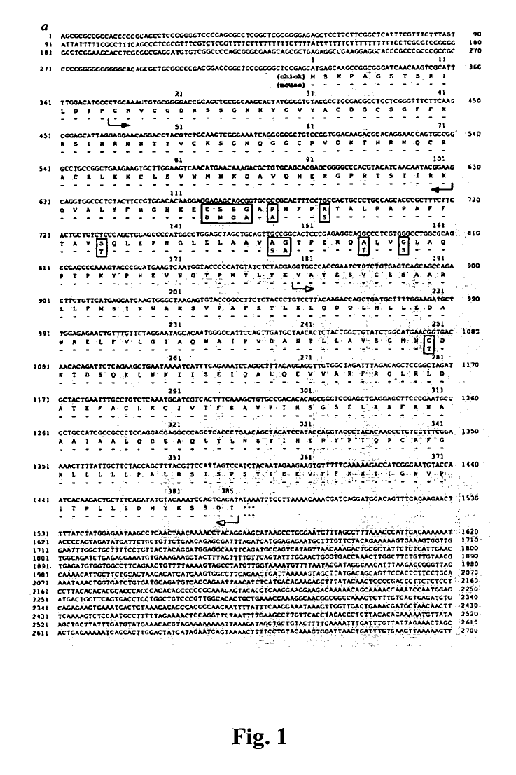

As used herein, a promoter region refers to a segment of DNA that controls transcription of DNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Exemplary TLX regulatory sequences can be found upstream of TLX coding sequences (see, for example, FIG. 1 and Yu, R. T., McKeown, M., Evans, R. M. & Umesono, K. “Relationship between Drosophila gap gene tailless and a vertebrate nuclear receptor Tlx” in Nature 370, 375-9 (1994)). Additional upstream regulatory sequences can readily be obtained using the above-described sequence(s) as a probe.

Compounds identified by the above-described methodology can be used in a variety of applications, e.g., methods for treating neurodegenerative diseases in a subject in need thereof; methods for reversing the reduction of neurogenesis from neural stem cells in a subject in need thereof; methods for promoting generation of neural stem cell populations in a subject in need thereof; methods for maintaining adult neural stem cells in an undifferentiated, proliferative state; methods for rescuing neural stem cell activity in a system in need thereof; methods for promoting neural stem cell activity in a system in need thereof; and the like. Such methods comprise inducing expression of TLX in said system, for example, by exposure to compound(s) identified by the above-described methodology.

As employed herein, “neurodegenerative diseases” embraces a variety of diseases, disorders or conditions, such as, for example, Alzheimer's disease, Parkinson's disease, Huntington's disease, amyotrophic lateral sclerosis, retinal degeneration, age-related hearing loss, mild cognitive impairment, dementia (including, for example, frontotemporal dementia, AIDS dementia, and the like), progressive supranuclear palsy, spinocerebellar ataxias, systemic senile amyloidosis, prion disease, scrapie, bovine spongiform encephalopathy, Creutzfeldt-Jakob disease, Gerstmann-Straussler-Scheinker syndrome, type II diabetes, adult onset diabetes, insulinoma, amyotropic lateral sclerosis, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, stroke and ischemia, and the like.

As employed herein, “reduction of neurogenesis from neural stem cells” refers to neural stem cell populations which have been depleted as a result of any of a variety of causes, e.g., age, disease, trauma, and the like.

As employed herein, “maintaining adult neural stem cells in an undifferentiated, proliferative state” refers to methods and/or conditions which maintain the ability of neural stem cells to proliferate, without promoting differentiation thereof.

As employed herein, “rescuing neural stem cell activity” refers to methods and/or conditions which facilitate restoration of desirable levels of neural stem cell activity.

As employed herein, “promoting neural stem cell activity” refers to methods and/or conditions which promote increased neural stem cell activity.

In accordance with a further embodiment of the present invention, there are provided methods for isolating adult neural stem cells from adult neural tissue, said methods comprising isolating β-gal-positive cells from TLX^{±} forebrain-derived tissue cultured on a LacZ substrate.

In accordance with yet another embodiment of the present invention, there are provided methods for producing adult neural stem cells from adult neural tissue, said methods comprising isolating β-gal-positive cells from TLX^{±} forebrain-derived tissue cultured on a LacZ substrate, and culturing same in suitable growth media.

In accordance with still another embodiment of the present invention, there are provided adult neural stem cells produced by the above-described methods.

In accordance with additional embodiments of the present invention, there are provided uses of over-expressed, purified TLX protein to isolate ligands (agonists or antagonists) by affinity purification. Compounds or molecules purified by such methods can then be characterized by standard structural analysis employing such techniques as mass-spectrometry, tandem mass-spectrometry, time of flight (tof) mass-spectrometry, nuclear magnetic resonance (NMR) or other quantitative analytical techniques.

In accordance with still further embodiments of the present invention, there are provided uses of over-expressed TLX ligand binding domain (LBD) as a dominant negative factor to block the action of full length protein (see, for example, FIG. 1; Yu, R. T., McKeown, M., Evans, R. M. & Umesono, K. “Relationship between Drosophila gap gene tailless and a vertebrate nuclear receptor Tlx” in Nature 370, 375-9 (1994); and Evans, R. M., “The Steroid and Thyroid Hormone Receptor Superfamily” in Science 240:889-895 (1988)). For example, the over-expressed LBD may form a non-productive heterodimer with the wild type protein or may bind co-factors needed to render the endogenous TLX active. Thus, it would act like a natural agonist or antagonist as it would as selectively alter the TLX signalling pathway. The LBD could be delivered to target cells in a variety of ways, e.g., by gene transfer, viral vector technology, and the like.

In accordance with a further embodiment of the present invention, there are provided uses of over-expressed TLX variant proteins as dominant positive (gain of function) or dominant negative (loss of function) factors to modulate TLX signaling and stem cell activity. Dominant positive variants include super-repressor forms of TLX such as fusions with other potent repressors such as the engrailed repressor domain. This variant should enhance the activity of the endogenous TLX pathway. Dominant negative variants include super-activator forms of TLX which can be achieved by fusions with transcriptional activators such as the herpes VP-16 peptide. This will antagonize the natural repression function of TLX to promote increased downstream signaling such as enhanced neurogenesis and glial genesis. Variants could be introduced into target cells in a variety of ways, e.g., by gene transfer, viral vector technology, and the like.

In accordance with yet another embodiment of the present invention, there are provided methods for treating neurodegenerative disease in a subject in need thereof. Such methods can be accomplished in a variety of ways, e.g., by introducing adult neural stem cells into the brain of said subject; by contacting brain cells of said subject with TLX; by exposing brain cells of said subject to TLX, and the like.

In accordance with still another embodiment of the present invention, there are provided methods for improving cognition in a subject in need thereof, said method comprising inducing expression of TLX in brain cells of said subject.

In accordance with a further embodiment of the present invention, there are provided methods for maintaining adult neural stem cells in an undifferentiated, proliferative state, said methods comprising contacting adult brain cells with TLX or exposing adult brain cells to TLX.

Thus, taking advantage of the β-galactosidase (β-gal) reporter that was knocked-into the TLX locus, the expression pattern of TLX was examined in adult brains of heterozygote mice. LacZ staining is observed to be distributed sparsely throughout the cortex, but reveals high-level but dispersed TLX expression in the subgranular layer of the DG and clustered expression in the subventricular zone (SVZ). Interestingly, these are the two major sites where adult NSCs reside (see Gage, F. H. “Neurogenesis in the adult brain” in J Neurosci 22, 612-3 (2002)).

Previous studies have suggested that nestin is a common marker of proliferating CNS progenitors (see Lendahl, U., Zimmerman, L. B. & McKay, R. D. “CNS stem cells express a new class of intermediate filament protein” in Cell 60, 585-95 (1990); and Reynolds, B. A., Tetzlaff, W. & Weiss, S. “A multipotent EGF-responsive striatal embryonic progenitor cell produces neurons and astrocytes” in J Neurosci 12, 4565-74 (1992)). β-gal and nestin staining on brain sections from adult TLX^{±} mice (see Lendahl, Zimmerman, & McKay, supra and Reynolds, Tetzlaff & Weiss, supra) reveal co-localization of β-gal and nestin in both the DG and SVZ, suggesting that TLX is expressed in adult neural stem/progenitor cells. An emerging concept contends that a subset of the stem cell pool corresponds to a repository of relatively quiescent cells that serve as the source for actively dividing cells (see Doetsch, F., Petreanu, L., Caille, I., Garcia-Verdugo, J. M. & Alvarez-Buylla, A. “EGF converts transit-amplifying neurogenic precursors in the adult brain into multipotent stem cells” in Neuron 36, 1021-34 (2002)). To examine if TLX-expressing cells represent the quiescent or dividing cells, β-gal and BrdU double-staining was performed with brain sections from BrdU-treated TLX^{±} mice. The analysis revealed that TLX expression corresponds to both BrdU-positive and -negative cells in the adult germinal zones (DG and SVZ).

Cell sorting was used to isolate TLX-expressing cells to determine their ability to proliferate, self-renew, and give rise to both neurons and glia. Using a fluorogenic LacZ substrate, β-gal-positive cells were isolated from TLX^{±} forebrains and cultured in N2 supplemented media with EGF, FGF, and heparin (see Allen, D. M. et al. “Ataxia telangiectasia mutated is essential during adult neurogenesis” in Genes Dev 15, 554-66 (2001)). Immunostaining confirmed β-gal expression in the FACS-sorted cells. The proliferation potential of TLX-positive cells was examined by BrdU-labeling of dividing cells. A 24-h BrdU treatment and subsequent immunostaining revealed that more than 98% of the β-gal-positive cells were BrdU- and nestin-positive.

Next the self-renewal capacity of TLX-expressing cells was tested using clonal analysis (see Taupin, P. et al. “FGF-2-responsive neural stem cell proliferation requires CCg, a novel autocrine/paracrine cofactor” in Neuron 28, 385-97 (2000)). Clonal populations were derived from single FACS-sorted, β-gal-positive cells treated with BrdU, followed by staining for β-gal, BrdU, and nestin. Twelve clones developed from a total of 28 single cells, eight of which reached more than 200 cells by day 12; another four reached this point by day 25. The clones were dissociated and single cells were plated for a second round of cloning. All the clones tested were also capable of secondary expansion. These results demonstrate that the TLX-expressing cells comprise a self-renewing population.

TLX cells derived from either primary or secondary clones were then tested to determine whether such cells could be induced to differentiate. Indeed, when examined for the expression of Tuj1 (a neuronal marker), GFAP (an astrocyte marker), or O4 (an oligodendrocyte marker), all three neural cell types with characteristic morphologies were generated upon differentiation, indicating that the TLX-expressing cells are multipotent.

In contrast, the cells isolated from the forebrains of TLX null littermates failed to proliferate under the same growth conditions. Immunostaining revealed that, while the TLX^{±} cells are nestin-positive and GFAP-negative, the TLX^{-/-} cells are nestin-negative but GFAP-positive, suggesting spontaneous astrocyte differentiation. In an attempt to rescue the proliferative defect, FACS-sorted adult TLX^{-/-} (LacZ/LacZ) cells were infected with a lentiviral vector expressing both TLX and GFP. The resulting infected cells were re-sorted based on the internal GFP marker. These infected and selected cells were observed to regain the ability to proliferate and can be clonally expanded. Remarkably, TLX expression leads to a significant restoration of cell proliferation, as revealed by Ki67 (a proliferative marker) and nestin staining, and reduced astrocyte differentiation, as revealed by GFAP staining. In contrast, cells that were infected with GFP control virus underwent spontaneous differentiation, as revealed by GFAP staining. Moreover, clonal analysis revealed that the lenti-TLX-expressing cells can be clonally expanded from single GFP-positive cells and are both nestin- and GFP-positive. Because the lenti-TLX is controlled by the constitutive CMV promoter, the rescued cells continue to proliferate even under differentiation conditions as expected. Together, these results demonstrate that TLX can rescue the undifferentiated, proliferative state of NSCs in vitro.

Next the transcriptional properties of TLX were examined to address how it might contribute to NSC maintenance. When fused to the GAL4 DBD, TLX strongly represses a luciferase reporter that is downstream of GAL4 DNA binding sites. The DNA binding domain of the yeast GAL4 protein comprises at least the first 74 amino acids thereof (see, for example, Keegan et al., Science 231:699-704 (1986)). Preferably, the first 90 or more amino acids of the GALA protein will be used, with the first 147 amino acid residues of yeast GAL4 being presently most preferred.

The GAL4 fragment employed in the practice of the present invention can be incorporated into any of a number of sites within TLX. For example, the GAL4 DNA binding domain can be introduced at the amino terminus of TLX, or the GAL4 DNA binding domain can be substituted for the native DNA binding domain of TLX, or the GAL4 DNA binding domain can be introduced at the carboxy terminus of TLX, or at other positions as can readily be determined by those of skill in the art. Thus, for example, a modified receptor protein can be prepared which consists essentially of amino acid residues 1-147 of GAL4, plus the ligand binding domain of TLX (i.e., containing the ligand binding domain only of said receptor (i.e., residues 180-385 as shown in FIG. 1), substantially absent the DNA binding domain and amino terminal domain thereof).

Exemplary GAL4 response elements are those containing the palindromic 17-mer:
5′-CGGAGGACTGTCCTCCG-3′(SEQ ID NO:6),
   such as, for example, 17MX, as described by Webster et al., in Cell 52:169-178 (1988), as well as derivatives thereof. Additional examples of suitable response elements include those described by Hollenberg and Evans in Cell 55:899-906 (1988); or Webster et al. in Cell 54:199-207 (1988).

Since histone deacetylases (HDACs) have been shown to mediate nuclear receptor transcriptional repression (see Nagy, L. et al. “Nuclear receptor repression mediated by a complex containing SMRT, mSin3A, and histone deacetylase” in Cell 89, 373-80 (1997)), a co-immunoprecipitation assay was used to see if TLX recruits any HDACs. Indeed, TLX interacts with HDAC1 and HDAC3 among the HDACs examined, suggesting that recruitment of HDACs is one of the mechanisms for TLX-mediated transcriptional repression.

In searching for downstream targets of TLX, a significant upregulation of GFAP, S100β, and aquaporin 4 (AQP4) expression was detected in the TLX mutant brains. TLX is expressed in the proliferating NSCs but switched off upon differentiation, whereas GFAP, S100β, and AQP4 are expressed upon differentiation. The case for direct regulation was strengthened when an analysis of the promoters of these genes revealed a consensus TLX binding site of AAGTCA. Gel shift analysis demonstrated that TLX could specifically bind to these sequences in vitro, suggesting that astrocyte-specific genes are direct downstream targets of TLX repression. To further confirm the repression by TLX, reporter assays using a GFAP promoter-driven luciferase reporter (GFAP-luc) (see Nakashima, K. et al. “Synergistic signaling in fetal brain by STAT3-Smad1 complex bridged by p300” in Science 284, 479-82 (1999)) were performed. Leukemia inhibitory factor (LIF) has been shown to induce astrocyte differentiation and GFAP expression (supra). Co-transfection of TLX leads to a significant repression (4.8-fold) of LIF-induced GFAP reporter activity, similar to the repression mediated by the dominant-negative STAT3 (supra; positive control).

To further establish the role of TLX in the repression of GFAP expression and astrocyte differentiation, NSCs were infected with the TLX-expressing retrovirus. Since it is under a constitutive CMV promoter, the viral TLX remains expressed upon differentiation, in contrast to the endogenous TLX that is down-regulated in differentiated cells. As expected, the expressions of GFAP and two other astrocyte-specific genes (s100β and aqp4) were significantly decreased in the viral TLX-expressing cells. Differentiation of these cells into GFAP-positive astrocytes was reduced proportionately. Immunostaining further revealed that NSCs with viral TLX expression failed to differentiate into GFAP+astrocytes upon treatment with LIF and BMP2, a condition favoring astrocyte differentiation. Instead, they continued expressing the neural progenitor marker nestin. Control NSCs infected with GFP virus differentiated into GFAP+ astrocytes and lost nestin expression under the same conditions. These results demonstrate the existence of a role for TLX in the repression of astrocyte differentiation.

The above analyses suggest a role for TLX in the maintenance of the undifferentiated, proliferative state of NSCs. Immunohistochemistry of the adult germinal zones revealed a dramatic reduction of nestin-positive cells in both the hippocampal DG and the SVZ of adult TLX null mice, indicating reduced neural precursors in the mutant neurogenic areas. Despite the hypotrophic nature of the mutant brains, increased GFAP staining was observed, consistent with the Northern analysis and suggesting increased glial differentiation in the mutant brains. TUNEL assays revealed no significant differences in cell death between wild type and mutant brains, suggesting a failure of ongoing cell proliferation or a very early window of cell death.

Next, the effects, if any, of TLX on cell proliferation in the adult brain was examined. BrdU labeling of adult mice was performed over a one-week period followed by immunohistochemistry. Intensive BrdU labeling was observed along the subgranular zone of the DG and in the SVZ of the TLX^{±} brains, whereas virtually no BrdU labeling was detected in the mutant hippocampus and SVZ. In contrast, as revealed by S100β staining, increased numbers of glial cells were observed in the mutant brains. These results demonstrate that TLX is essential in the maintenance of the undifferentiated, proliferative state of stem cells in adult brains.

The existence of adult NSCs raises a question: what are the molecular determinants of this unique cell population? The characterization of TLX provided herein suggests that it is one of the key regulators that act by controlling the expression of a network of target genes to establish the undifferentiated and self-renewable state of NSCs. The expression of TLX in scattered cells throughout the cortex suggests that it may play another role in addition to its function in the germinal zone. Furthermore, the dominant role in stem cell maintenance that TLX plays in the adult is clearly different from a seemingly more modest role in development. Finally with regards to the persistence of a NSC in the absence of TLX in the adult, the fact that the adult rescue experiment works at all indicates either that a dormant progenitor/stem cell population exists or that TLX expression alone can recover a stem cell state in a cell population obtained from the adult germinal region. The characterization of the TLX-expressing cells provides a means to elucidate the molecular and cellular mechanisms for stem cell proliferation and differentiation. Furthermore, the use of β-gal-based FACS facilitates the isolation of NSCs from adult brains, which will make it possible to explore clinical applications for transplantability and molecular engineering associated with the treatment of neurodegenerative diseases and brain injuries.

The invention will now be described in greater detail with reference to the following non-limiting examples.

### EXAMPLE 1

### Immunocytochemistry and Quantification

LacZ staining was carried out on 40-μm free-floating sections or with cultured cells as described (see Lie, D. C. et al. “The adult substantia nigra contains progenitor cells with neurogenic potential” in J Neurosci 22, 6639-49 (2002), and Taupin, P. et al. “FGF-2-responsive neural stem cell proliferation requires CCg, a novel autocrine/paracrine cofactor” in Neuron 28, 385-97 (2000)). Primary antibodies included: BrdU (1:250, rat; Accurate), nestin (1:1000, mouse; Pharmingen), Tuj1 (1:1000, mouse; Bavco), S100β (1:500, rabbit; Sigma), GFAP (1:500, guinea pig; Advance Immuno), 04 (1:3, mouse IgM; O. Boegler), β-gal (1:2000, rabbit; Cortex), and Ki67 (1:1000, rabbit, Novocastra). BrdU-labeled cells were treated in 1M HCL at 37° C. for 30 min and visualized using confocal microscopy (Bio-Rad, Richmond, Calif.). Quantitative studies were based on four or more replicas. The DG subgranular areas were traced using semi-automatic stereology (Stereolnvestigator, MicroBrightfield) and volumes were determined using the areas multiplied by the thickness of the sections. Cell densities were calculated by dividing cell numbers by the volume. Statistical analysis was performed using Microsoft Excel. For LacZ staining, 40-μm frozen sections were fixed with glutaraldehyde and paraformaldehyde and stained in X-gal (5-bromo-4-chloro-3-indolyl-β-D-galactopyranoside, BRL) solution containing 20 mM K-ferricyanide, 20 mM ferrocyanide, 0.01% Na-deoxycholate, 0.02% NP-40, and 2 mM MgCl2. 40× or 10× objectives were used for the images. Niss1 staining was performed using 0.25% Cresyl Violet on 40-μm coronal sections.

### EXAMPLE 2

### Preparation and Culture of Adult Neural Stem/Progenitor Cells

Mouse forebrains were minced with scalpels and digested in PPD solution (Papain, Dispase II, and DNase I). Cells were isolated using Percoll gradients 18 and cultured in DMEM/F12 with 2.5 mM L-glutamine and N2 supplemented with EGF (20 ng/ml), FGF-2 (20 ng/ml), and heparin (50 ng/ml) (see Allen, D. M. et al. “Ataxia telangiectasia mutated is essential during adult neurogenesis” in Genes Dev 15, 554-66 (2001)). Cells were collected using β-gal-based FACS (Molecular Probes). For differentiation, cells were exposed to N2-supplemented media containing 5 μM forskolin and 0.5% FBS for one week. Rat NSCs were cultured in N2-supplemented media containing FGF-2 (20 ng/ml) as previously described (see Palmer, T. D., Markakis, E. A., Willhoite, A. R., Safar, F. & Gage, F. H. “Fibroblast growth factor-2 activates a latent neurogenic program in neural stem cells from diverse regions of the adult CNS” in J Neurosci 19, 8487-97 (1999)). Neural differentiation was initiated with N2-supplemented media containing 1 μM retinoic acid and 0.5% FBS for 4 days. For astrocyte differentiation, cells were cultured in N2 media containing 50 ng/ml LIF, 50 ng/ml BMP2, and 1% FBS. Clonal analysis was performed in conditional CCg media (see Taupin, P. et al. “FGF-2-responsive neural stem cell proliferation requires CCg, a novel autocrine/paracrine cofactor” in Neuron 28, 385-97 (2000)). β-gal-positive cells were plated in 96-well plates. Cells were counted four hours after plating. Wells containing single cells were monitored continuously until they reached >200 cells.

### EXAMPLE 3

### In vivo BrdU Labeling

Eight-week-old mice were injected intraperitoneally once daily with BrdU (50 mg/kg) over a 12-day period. Brains were fixed one day after the last injection and processed for immunostaining as previously described (see Lie, D. C. et al. “The adult substantia nigra contains progenitor cells with neurogenic potential” in J Neurosci 22, 6639-49 (2002)). Alternatively, BrdU (1 mg/ml) was administered to mice via drinking water for two weeks and brain tissues were processed for immunostaining.

### EXAMPLE 4

### Northern Blot Analysis and Gel Shift Assay

Total RNA from cultured cells or brain tissues was isolated using Trizol reagent (Life Technologies). Northern blot analyses were carried out as previously described (see Shi, Y. et al. “Sharp, an inducible cofactor that integrates nuclear receptor repression and activation” in Genes Dev 15, 1140-51 (2001)). Gel shift assay was performed as previously described (see Yu, R. T., McKeown, M., Evans, R. M. & Umesono, K. “Relationship between Drosophila gap gene tailless and a vertebrate nuclear receptor Tlx” in Nature 370, 375-9 (1994)) using in vitro translated TLX and 32P-labeled GFAP probes.

### EXAMPLE 5

### Transient Transfection Assays and Immunoprecipitation Analysis

CV-1 cells were transiently transfected as previously described (see Shi, Y. et al. supra). Adult rat neural progenitor cells were transfected using Transit-LT (Mirus). Luciferase activity of each sample was normalized by β-gal activity in CV-1 and Renilla luciferase activity in progenitor cells. Transfections were done in triplicate at least three times. Immunoprecipitation analysis was performed as previously described (see Shi, Y. et al., supra) by transfecting 293 cells with Flag-tagged HDACs and HA-tagged TLX. The lysates were immunoprecipitated with Flag-specific antibody and immunoblotted with HA-specific antibody.

### EXAMPLE 6

### Viral Production and Infection

A TLX-expressing retrovirus was produced using either an NIT vector and 293gp cells (see Palmer, T. D., Markakis, E. A., Willhoite, A. R., Safar, F. & Gage, F. H. “Fibroblast growth factor-2 activates a latent neurogenic program in neural stem cells from diverse regions of the adult CNS” in J Neurosci 19, 8487-97 (1999)) or the pMY vector (see Misawa, K. et al. “A method to identify cDNAs based on localization of green fluorescent protein fusion products” in Proc Natl Acad Sci USA 97, 3062-6 (2000)) and Plat-E cells (see Morita, S., Kojima, T. & Kitamura, T. “Plat-E: an efficient and stable system for transient packaging of retroviruses” in Gene Ther 7, 1063-6 (2000)). The TLX-expressing lentivirus was produced using pCSC vector (see Miyoshi, H., Blomer, U., Takahashi, M., Gage, F. H. & Verma, I. M. “Development of a self-inactivating lentivirus vector” in J Virol 72, 8150-7 (1998)) and 293T cells. The transgene within the NIT vector is expressed from a minimal CMV promoter containing 6 tetracycline operators. The transgene in the pMY or pCSC vector is expressed from a CMV promoter and upstream of an IRES GFP marker. The NSCs were infected by incubating with the virus and polybrene (2 μg/ml, sigma). Cells expressing NIT-TLX were selected with G418 (400 μg/ml). Cells expressing pMY-TLX and pCSC-TLX were sorted by the IRES GFP marker.

### EXAMPLE 7

### TLX as a Dominant Negative Factor

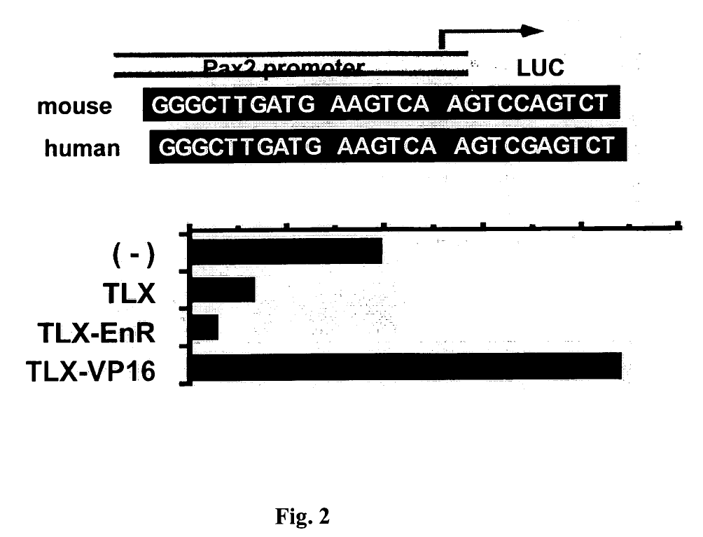

A construct comprising a Pax2 promoter operatively associated with a reporter gene (e.g., luciferase) was exposed to TLX alone, TLX-EnR and TLX-VP (see Yu, R. T. et al. “The orphan nuclear receptor Tlx regulates Pax2 and is essential for vision” in Proc Natl Acad Sci USA 97, 2621-5 (2000)). In the presence of TLX alone or TLX-EnR, the Pax2 promoter is repressed, as demonstrated by the reduced expression of reporter (see FIG. 2). Conversely, in the presence of TLX-VP, expression via the Pax2 promoter is de-repressed, demonstrating the dominant negative properties of TLX-VP.

While the invention has been described in detail with reference to certain preferred embodiments thereof, it will be understood that modifications and variations are within the spirit and scope of that which is described and claimed.

That which is claimed is:

1. A method for screening compounds to determine those which modulate the activity of TLX, said method comprising contacting a test cell with one or more test compounds, and assaying for evidence of transcription of reporter by said test cells,
wherein said test cell expresses TLX and contains a reporter construct comprising a TLX-response element operatively linked to a reporter gene.

2. A method for relieving TLX-mediated transcription repression and/or inducing processes mediated by TLX, said method comprising conducting said process in the presence of a compound identified by the method of claim 1.

3. The method of claim 2 further comprising conducting said process in the further presence of at least one inhibitor of a co-repressor.

4. A method for inhibiting processes mediated by TLX, said method comprising conducting said process in the presence of a compound identified by the method of claim 1.

5. A method for promoting stem cell differentiation in a system in need thereof, said method comprising contacting said system with a compound identified by the method of claim 1.

6. A method for promoting stem cell differentiation in a system in need thereof, said method comprising blocking expression of TLX in said system.

7. A method for screening compounds to determine those which modulate the expression of TLX, said method comprising contacting a test cell with one or more test compounds, and assaying for evidence of transcription of reporter by said test cells,
wherein said test cell contains a reporter construct comprising a TLX promoter operatively linked to a reporter gene.

8. A method for treating neurodegenerative disease in a subject in need thereof, said method comprising inducing expression of TLX in brain cells of said subject.

9. A method for reversing the reduction of neurogenesis from neural stem cells in a subject in need thereof, said method comprising inducing expression of TLX in brain cells of said subject.

10. A method for promoting generation of neural cell populations in a subject in need thereof, said method comprising inducing expression of TLX in brain cells of said subject.

11. A method for maintaining adult neural stem cells in an undifferentiated, proliferative state, said method comprising inducing expression of TLX in adult brain cells.

12. A method for rescuing neural stem cell activity in a system in need thereof, said method comprising inducing expression of TLX in said system.

13. A method for promoting neural stem cell activity in a system in need thereof, said method comprising inducing expression of TLX in said system.

14. A method for isolating adult neural stem cells from adult neural tissue, said method comprising isolating β-gal-positive cells from TLX^{±} forebrain-derived tissue cultured on a LacZ substrate.

15. An adult neural stem cell produced by the method of claim 14.

16. A method for producing adult neural stem cells from adult neural tissue, said method comprising isolating β-gal-positive cells from TLX^{±} forebrain-derived tissue cultured on a LacZ substrate, and culturing same in suitable growth media.

17. An adult neural stem cell produced by the method of claim 16.
